(19) United States
(12) Patent Application Publication
(10) Pub. No.: US 2019/0001073 A1
(43) Pub. Date: Jan. 3, 2019

(54) SURFACE HARDENED INJECTION NEEDLE AND METHOD OF PRODUCING SUCH

(71) Applicant: Novo Nordisk A/S, Bagsvaerd (DK)

(72) Inventors: Soeren Iversen, Herlev (DK); Jacqueline Trosborg, Kongens Lyngby (DK)

(21) Appl. No.: 15/737,834

(22) PCT Filed: Jun. 22, 2016
(86) PCT No.: PCT/EP2016/064348

§ 371 Date: Dec. 19, 2017

(30) Foreign Application Priority Data
Jul. 6, 2015 (EP) 15175453.8
Aug. 18, 2015 (EP) 15181380.5

(51) Int. Cl. A61M 5/32; A61L 31/02; C23C 8/32; C23C 8/02

(52) U.S. Cl. CPC A61M 5/329; A61M 5/3286; A61L 31/022; A61M 2207/00; A61L 31/028; C23C 8/32; C23C 8/02; A61L 31/024

(57) ABSTRACT

A medical injection needle (1) having a metallic needle body (2) comprising an axially extending wall (3), a first end portion (4), a second end portion (6), and a flow path (7) providing for fluid communication between the first end portion (4) and the second end portion (6) along the axially extending wall (3), wherein at least a portion of the metallic needle body (2) comprises a hardened surface layer (10, 20) in which carbon atoms and nitrogen atoms are deposited.

### FIELD OF THE INVENTION

The present invention relates to medical needles in general and more specifically to injection needles for penetration of human skin and dermal or hypodermal delivery of therapeutic substance.

### BACKGROUND OF THE INVENTION

Injection needles are widely used in the medical area to deliver medicaments to specific body sites. For example, within the treatment of diabetes mellitus pen needles are oftentimes used with injection pens for subcutaneous administration of various glucose regulating agents. A pen needle comprises an injection needle and a needle hub having means for attachment to an injection pen. The injection needle is typically fixed to the needle hub so as to enable skin penetration by the one needle end and penetration of a cartridge septum by the other needle end.

Such pen needles should ideally be used only once, particularly to minimise the risks of contamination and needle induced tissue damage. The latter is destined to occur when the same injection needle is used for multiple skin penetrations, as hook formation at the needle tip is an inevitable result of repeated insertion through skin. Even though pen needle manufacturers tout a single use policy a Diabetes Patient Market Study under Roper Global Diabetes Programme has shown that an average patient reuses her/his injection needle approximately six times. It is therefore desirable to design an injection needle having a large resistance to hook formation.

The overall increased demand for thinner injection needles and higher flow rates entails smaller wall-thicknesses of the needle tubes and thereby weaker needle tips. Previously, attempts have been made to minimise the hooking tendency by modifying the geometry of the needle tips, replacing the conventional three grinds with one or two blunter grinds. However, this increases both the pressure required to pierce the skin and the discomfort of inserting the needle.

Needle tubes may be hardened to increase their resistance to bending, but hardening must be performed with due account to the crucial requirement of avoiding stiffness related breakage of an injection needle during use. So, whereas hardening conventionally enhances the structural stability of the needle shaft it also entails an increased risk of needle breakage.

U.S. Pat. No. 2,170,844 (Van Note) concerns a method of hardening tantalum articles being subject to wear from their specific use and discloses a production of such hardened articles by heating in an atmosphere of less than atmospheric pressure, to a low temperature in an atmosphere of hardening gas for an extended period of time, and subsequently, in a vacuum heating chamber, raising the temperature above the initial heating temperature and maintaining the same until the absorbed gas has been diffused and distributed through the body to the desired degree. By control of the initial heating as well as the subsequent soaking period the articles may obtain a graduated hardness varying from the outside in. While hypodermic needles are in fact cursorily mentioned as an exemplary type of article to undergo the disclosed process steps the linear nature of the soaking process appears to severely limit the obtainable degree of hardness variation through a thin-walled such specimen.

US 2012/0111456 (Expanite A/S) discloses a method of activating an article of passive ferrous or non-ferrous metal in order to remove or transform the superficial oxide layer prior to case hardening by e.g. nitrocarburising. The method appears to be developed for large scale articles. There is no teaching or indication of the method being applicable to an article having a wall-thickness in the dimension of an injection needle.

### SUMMARY OF THE INVENTION

It is an object of the invention to eliminate or reduce at least one drawback of the prior art, or to provide a useful alternative to prior art solutions.

In particular, it is an object of the invention to provide an injection needle, especially a thin-walled injection needle, exhibiting a reduced tendency to hooking or bending of the needle tip.

It is a further object of the invention to provide such an injection needle having a low risk of stiffness related breakage.

It is also an object of the invention to devise a method for providing a surface hardened injection needle.

In the disclosure of the present invention, aspects and embodiments will be described which will address one or more of the above objects and/or which will address objects apparent from the following text.

An injection needle embodying the principles of the invention comprises a metallic needle body in a portion of which both carbon and nitrogen are dissolved. The carbon and the nitrogen together establish a double hardened zone in the metal which facilitates a steep hardness gradient and thereby provides for the combination of a hard needle body surface and a much softer needle body core. This combination is desirable as it enables the production of an injection needle which is more resistant to repeated skin insertions, yet still sufficiently flexible to avoid breakage.

In one aspect the invention provides an injection needle as defined in claim 1.

Thus, a medical injection needle may be provided, which injection needle comprises a metallic needle body having a wall, e.g. a cylindrical wall, extending along a longitudinal axis, a first end portion, and a second end portion. The first end portion and the second end portion are in fluid communication via one or more flow paths, i.e. fluid flow between the first end portion and the second end portion is enabled within the wall, e.g. through a lumen therein, and/or along an exterior portion of the wall. At least a portion of the metallic needle body comprises a surface layer in which carbon atoms and nitrogen atoms are deposited. This surface layer is hardened and hence provides for a portion of the metallic needle body being more resistant to physical deformation than the remaining portion of the injection needle.

The hardened surface layer may comprise an inner layer in which predominantly carbon atoms are deposited and an outer layer in which predominantly nitrogen atoms are deposited. Thereby, the nitrogen atoms increase the surface hardness, while the carbon atoms bridge the gap to a softer core, enabling a steep hardness gradient in the material that may result in a 4-5 times greater hardness of the exterior surface than of the core.

The hardened surface layer may have a radial extent from the outer surface of the metallic needle body and inward (i.e. thickness) which does not exceed 25 μm. For example, the radial extent may be in the range of [10 μm; 25 μm]. This is particularly relevant in relation to thin-walled needle specimens, which can then be provided with both a hardened exterior surface and a hardened interior surface while still maintaining a much softer core.

The hardened surface layer may even have a radial extent which does not exceed 10 μm, for example lying in the range [5 μm; 10 μm]. This is particularly relevant in relation to extrathin-walled needle specimens.

The at least a portion of the metallic needle body comprising the hardened surface layer may comprise the first end portion. In that respect the first end portion may be the portion of the needle which comprises a sharpened tip configured for penetration of a human skin. During insertion of the needle into the skin in connection with a drug administration procedure the first end portion is exposed to mechanical contact forces from the interaction with the various skin layers, and given the miniscule geometry of the tip repeated insertions greatly increases the risk of tip deformation. A surface hardening of the first end portion results in the tip becoming more resistant to wear and thus capable of enduring multiple skin insertions without bending or hooking.

The metallic needle body, specifically the wall, the first end portion and the second end portion together, may comprise a radially outwardly oriented surface and a radially inwardly oriented surface. The hardened surface layer may be present along at least a portion of at least one of the radially outwardly oriented surface and the radially inwardly oriented surface. The metallic needle body may thus selectively be hardened along an exterior surface, along an interior surface, or along both an exterior surface and an interior surface.

The wall connecting the first end portion and the second end portion may be tubular, and the injection needle may be normal-walled, thin-walled, or even extra-thin-walled, the tubular wall thus, depending on the gauge size, e.g. having a thickness in the range [20 μm; 500 μm], such as in the range [20 μm; 180 μm], in the range [25 μm; 80 μm], or in the range [25 μm; 50 μm]. Alternatively, the wall connecting the first end portion and the second end portion may be conical or partly conical.

In particular embodiments of the invention a first hardened surface layer is present along at least a portion of the radially outwardly oriented surface and a second hardened surface layer is present along at least a portion of the radially inwardly oriented surface, and the metallic needle body has a core section positioned between the first hardened surface layer and the second hardened surface layer. The hardness of the at least a portion of the radially outwardly oriented surface is 3-5 times greater than the hardness of the core section.

The injection needle may form part of an injection needle assembly, such as a so-called pen needle assembly. In that case the metallic needle body is fixedly arranged in a needle hub element being adapted for attachment onto a drug delivery device, e.g. an injection device. A portion of the wall may thereby extend distally from the needle hub element, defining a front needle portion for insertion into skin. An opposite portion of the wall may extend proximally from the needle hub element, defining a back needle portion for insertion into a drug reservoir, e.g. through a membranous closure.

The hardened surface layer may be present only along a surface of the metallic needle body which is further away from the needle hub element than 1.5 mm. Thereby, the area of the metallic needle body which is closest to the point of fixation is not surface hardened, increasing the flexibility of the injection needle in that area to safely accommodate the radial deflections of the first end portion which may occur during normal handling of the injection needle assembly and/or insertion of the front needle portion through the skin.

In another aspect of the invention a method of hardening an injection needle is provided, the method comprising step (i) bringing at least a portion of the injection needle having a first temperature (T₁) between 200° C. and 500° C. in contact with a gaseous substance derived from a compound containing nitrogen and carbon and having a second temperature (T₂) between 200° C. and 500° C., step (ii) subsequent to step (i) bringing the at least a portion of the injection needle in contact with a carbon gas having a third temperature (T₃) which is at least as high as the first temperature and lower than 500° C., and step (iii) subsequent to step (ii) bringing the at least a portion of the injection needle in contact with a nitrogen gas having a fourth temperature (T₄) which is at least as high as the first temperature and lower than 500° C. Eventually, the injection needle is cooled to room temperature. The cooling may e.g. be carried out in an atmosphere of Argon gas in less than 10 minutes. This method may be used to produce an injection needle as described in the above.

A passive metallic surface having the first temperature and being brought in contact with a gas evolving from a nitrogen/carbon containing compound having the second temperature will become activated and thereby prepared for receiving carbon and nitrogen atoms in the course of the subsequent method steps. This enables a relatively fast nitrocarburising surface hardening treatment of injection needles made of e.g. stainless steel.

For example, the method may comprise heating an injection needle to a first temperature, T₁, between 200° C. and 500° C., heating a compound containing nitrogen and carbon to a second temperature, T₂, between 200° C. and 500° C., thereby producing an activating gaseous substance, bringing at least a portion of the heated injection needle in contact with the activating gaseous substance to thereby obtain an activated needle portion, bringing the activated needle portion in contact with a carbon gas having a third temperature, T₃, being at least as high as T₁ yet smaller than 500° C., and subsequently bringing the activated needle portion in contact with a nitrogen gas having a fourth temperature, T₄, being at least as high as T₁ yet smaller than 500° C.

The various process temperatures are kept below 500° C. to prevent formation of nitrides or carbides, which may otherwise affect the corrosion resistance of the metal. The third temperature and the fourth temperature may be identical, or at least substantially identical.

The injection needle may comprise a cylindrical wall extending along a longitudinal axis, a first end portion, and a second end portion.

The method may further comprise electropolishing and/or grinding the first end portion prior to step (i). The electropolishing pre-process step may e.g. be employed in order to provide a conically tapering first end portion. Grinding may be carried out to provide a sharpened tip. Thereby, the injection needle may be brought to its final shape before the surface hardening process is carried out, providing for a very wear resistant first end portion, as post-treatment can be avoided.

The method may further comprise electropolishing and/or grinding the first end portion subsequent to step (iii). The electropolishing post-process step may e.g. be employed in order to shape or deburr the first end portion. Post-process grinding may be chosen to reduce brittleness of the tip.

The method may further comprise shielding a portion of the injection needle during steps (i)(iii) to thereby obtain one or more distinct areas having properties which are different from those of the areas having undergone the surface hardening process. Specifically, the method may further comprise covering a portion of the metallic needle body with a shield prior to step (i). Even further, the method may comprise removing the shield from the metallic needle body subsequent to step (iii). The shield may be a physical cover in the form of e.g. a skeletal mask applied over the metallic needle body, or a bag structure transpierced by a portion of the injection needle. Depending on the specific type of shield the removal of the shield from the metallic needle body subsequent to step (iii) may be carried out by the manufacturer or, potentially, by the user of the final product.

The physical cover may be a separate entity or it may form part of a chamber in which the at least a portion of the injection needle and the activating gaseous substance and/or at least one of the carbon gas and the nitrogen gas interact. Alternatively, the shield may be applied directly onto a portion of the metallic needle body, such as by adhesion. Specifically, Cu paste may be applied to one or more predefined areas of the metallic needle body prior to step (i) and removed therefrom subsequent to step (iii).

In particular embodiments of the invention, the method comprises applying the shield to cover the metallic needle body at least in an area which is between 1.5 mm and 9 mm from the sharpened tip. Thereby, the first end portion may undergo the surface hardening process while the remaining portion of the injection needle, or at least the portion of the cylindrical wall which extends from the first end portion to a contemplated point of attachment to a needle hub element, may avoid the surface hardening and thereby retain its original surface structure and flexibility. It is emphasized, though, that any desired portion, or portions, of the metallic needle body may undergo the present surface hardening treatment, while all other portions are exempted therefrom.

In the present context, a hardened surface layer in which carbon atoms and nitrogen atoms are deposited is a surface layer to which carbon atoms and nitrogen atoms have been artificially added, by a nitrogen and carbon adding process (such as a nitrocarburising process), as opposed to a material layer exhibiting any natural presence of carbon atoms and nitrogen atoms. A hardened surface layer in accordance with various exemplary embodiments of the invention may e.g. exhibit a local content of both carbon and nitrogen which exceeds 0.3%, 0.4%, 0.5%, 1%, 1.5%, 2% or 3%, respectively. Alternatively, the hardened surface layer may e.g. exhibit a local content of carbon which exceeds 0.4% or 0.5% and a local content of nitrogen which exceeds 0.25% or 0.3%.

It is understood that the term “injection needle” covers both a needle structure which is processed to a degree allowing for a safe use of it as a medical injection needle as well as a tubular, or conical, structure which does not yet have the shape and/or finish of a desired final injection needle product. One example of such a structure is a needle tubing which is not of the desired length. Another example is a needle-like structure which is not ground.

In the present specification, reference to a certain aspect or a certain embodiment (e.g. “an aspect”, “a first aspect”, “one embodiment”, “an exemplary embodiment”, or the like) signifies that a particular feature, structure, or characteristic described in connection with the respective aspect or embodiment is included in, or inherent of, at least that one aspect or embodiment of the invention, but not necessarily in/of all aspects or embodiments of the invention. It is emphasized, however, that any combination of the various features, structures and/or characteristics described in relation to the invention is encompassed by the invention unless expressly stated herein or clearly contradicted by context.

The use of any and all examples, or exemplary language (e.g., such as, etc.), in the text is intended to merely illuminate the invention and does not pose a limitation on the scope of the same, unless otherwise claimed. Further, no language or wording in the specification should be construed as indicating any non-claimed element as essential to the practice of the invention.

### BRIEF DESCRIPTION OF THE DRAWINGS

In the following the invention will be further described with references to the drawings, wherein

FIG. 1 is a longitudinal section view of an injection needle according to an embodiment of the invention,

FIG. 2 is a cross-sectional view of the injection needle of FIG. 1,

FIG. 3 shows an injection needle having a deformed tip,

FIG. 4 is a sketch of the surface structure of an injection needle having undergone a surface hardening treatment according to an embodiment of the invention,

FIG. 5 is a cross-sectional micrograph of a wall portion of an injection needle having undergone a surface hardening treatment according to an embodiment of the invention,

FIG. 6 is a graphical representation of the hardness variation through a wall portion of an injection needle according to an embodiment of the invention,

FIGS. 7-9 show different processes for obtaining an injection needle according to different embodiments of the invention, and

FIG. 10 is a longitudinal section view of a pen needle assembly including an injection needle according to an embodiment of the invention.

In the figures like structures are mainly identified by like reference numerals.

### DESCRIPTION OF EXEMPLARY EMBODIMENTS

When in the following relative expressions, such as “upper” and “lower”, are used, these refer to the appended figures and not necessarily to an actual situation of use. The shown figures are schematic representations for which reason the configuration of the different structures as well as their relative dimensions are intended to serve illustrative purposes only.

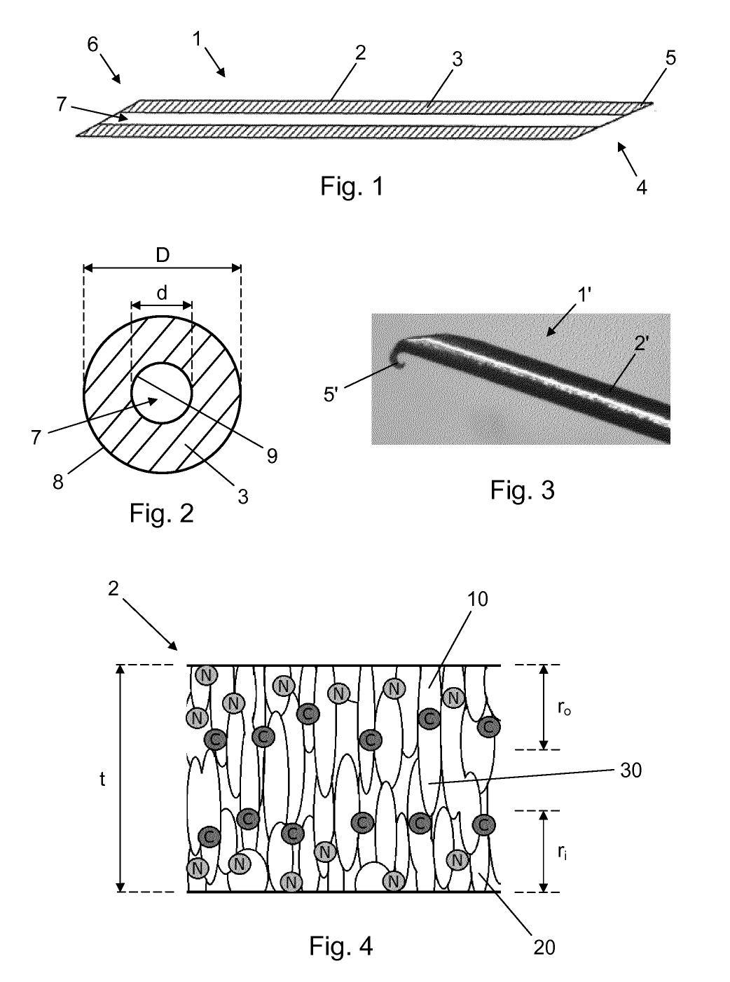

FIG. 1 is a longitudinal section view of an injection needle 1 having an elongated metallic needle body 2. The needle body 2 comprises a tubular wall 3 extending between a subject end portion 4 and a reservoir end portion 6 and defining a lumen 7 for conveying fluid. The subject end portion 4 is processed to provide a sharpened tip 5 for easy and virtually painless insertion through a human skin.

FIG. 2 shows the injection needle 1 in cross-section. It is seen that the tubular wall 3 has an external pipe diameter, D, and the lumen 7 has a diameter, d. The needle body 2 is further defined by a radially outwardly oriented exterior surface 8 and a radially inwardly oriented interior surface 9.

The injection needle 1 may be formed in accordance with any conventional method, e.g. seamless tubing in which a solid steel bar is extruded in a repeated cold drawing process to create a throughgoing bore. Work hardening as a natural consequence of the cold drawing process reduces ductility so the formed tube is normally annealed between drawing operations to increase ductility and prevent the material from becoming brittle. An alternative method, known as welded tubing, forms a flat metal strip into a tubular shape and uses a high energy source to melt the metal locally at the edges of the open seam to create a fusion juncture. The thus produced weld line is typically cold worked locally to exhibit similar properties as the base metal and annealed for stress relief, recrystallization and complete homogenizing.

A number of such formed tubes may then be assembled in a tube bundle and cut to obtain a final desired length and each tube is ground to provide either one or two sharpened tips.

Optionally, the tubes are electropolished to provide a conical exterior shape. Regardless of the specific forming method the final needle product must possess specific material and structural properties which allow it to penetrate a skin barrier without deflecting or breaking.

FIG. 3 shows a picture of an injection needle 1′ with a needle body 2′ and a needle tip 5′. The injection needle 1′ has been used multiple times and the picture shows the accumulated deformation of the needle tip 5′. The exhibited phenomenon is known as hooking.

FIG. 4 is a magnified sketch of the atomic structure of a portion of the needle body 2 following a surface hardening treatment according to an embodiment of the invention. The figure shows a segment of the tubular wall 3 in cross-section and the atomic arrangement across the entire wall thickness, t=½(D−d), is sketched. The original material structure is still present in a core layer 30, but an outer hardened surface layer 10 has been established at the exterior surface 8, while an inner hardened surface layer 20 has been established at the interior surface 9. Both hardened surface layers 10, 20 are characterised by an expansion of the material structure stemming from deposited nitrogen and carbon atoms.

The outer hardened surface layer 10 has a radial extent, or thickness, rₒ, and the inner hardened surface layer 20 has a radial extent, or thickness, rᵢ, which are pendent on specific process parameters, including potential shielding times, where parts of the interior surface 9 and/or the exterior surface 8 are covered up by a physical shield. In FIG. 4, rₒ and rᵢ are practically identical. However, it is noted that the thickness of the respective hardened surface layers 10, 20 may be designed by modification of certain process parameters so as to optimise the material and structural properties of a given injection needle type, and this optimisation needs not imply equal thicknesses. For thin-walled injection needles values of rₒ and rᵢ may, for example, lie in the range [10 μm; 25 μm], while for very-thin-walled injection needles values of rₒ and rᵢ may, for example, lie in the range [5 μm; 10 μm].

A surface hardening process according to an embodiment of the invention enables a particular formation of the deposited nitrogen and carbon atoms which comprises an inner layer in which predominantly carbon atoms are present and an outer layer in which predominantly nitrogen atoms are present, as also indicated in FIG. 4. The nitrogen atoms primarily increase the surface hardness while the carbon atoms bridge the gap to the softer core layer 30. Thereby, a smooth hardness profile exhibiting a steep gradient towards the core layer 30 is obtained. The steep gradient enables the realisation of very thin hardened surface layers 10, 20 prerequisite for thin-walled specimens of the order of magnitude of hypodermic injection needles.

Suitable materials for surface hardening according to the present invention are e.g. stainless steel grades 201, 301 and 304, PH steels, maraging stainless steels, and maraging stainless steels with cobalt. In particular embodiments of the invention the needle body 2 is made of austenitic stainless steel of the type X11CrNiMnN19-8-6 (ISO 15510:2014(E)).

### Example

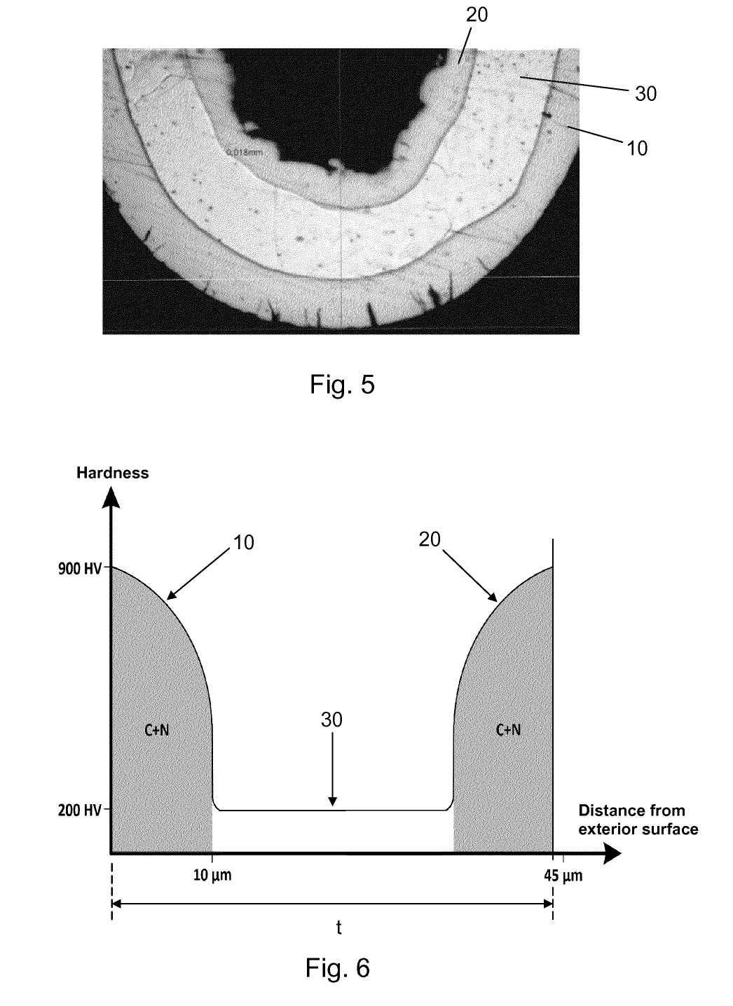

A plurality of 32G cannulas made of 304 stainless steel is heated to a temperature of 490° C. in a reducing gas, H₂. The supply of H₂ is cut off and the passive ferrous metal surface is activated by heating a urea compound to a temperature of 490° C. and bringing the heated urea compound in contact with the cannulas. The temperature is kept below 500° C. to avoid the formation of nitrides and carbides, which might otherwise affect the corrosion resistance of the metal. Once the surface has been activated the supply of urea is cut off and replaced with a supply of carbon gas for approximately 1 hour. Then the supply of carbon gas is interrupted and nitrogen gas is supplied to the metal surfaces for approximately 4 hours. The thereby established N/C concentrated surface layer comprises an innermost layer in which predominantly carbon atoms are deposited and an outermost layer in which predominantly nitrogen atoms are deposited. The cannulas are finally cooled to room temperature in an atmosphere of argon gas in less than 10 minutes. FIG. 5 is a microscopic scale capture of a segment of a cannula wall, in cross-section, clearly identifying the various treated and nontreated layers. The maximum total thickness of the inner hardened surface layer 20 and the outer hardened surface layer 10, respectively, was 18 μm.

FIG. 6 is a graphical estimation of a hardness profile obtained by another exemplary embodiment of the invention. The graph shows the material hardness of the needle body 2, measured according to the Vickers standard, as a function of the distance from the exterior surface 8. The hardness of the core layer 30 is approximately 4.5 times lower than that of the exterior surface 8, respectively the interior surface 9, and the radial extent of each of the outer hardened surface layer 10 and the inner hardened surface layer 20 is only about 10 μm. This provides for a much increased resistance of the treated surfaces to mechanical impact while a certain flexibility of the core of the needle body 2 is maintained. Because the radial extent of the respective hardened surface layers 10, 20 is so small the volume of the core layer 30 is substantial by comparison, reducing any tendency of the injection needle 1 to break.

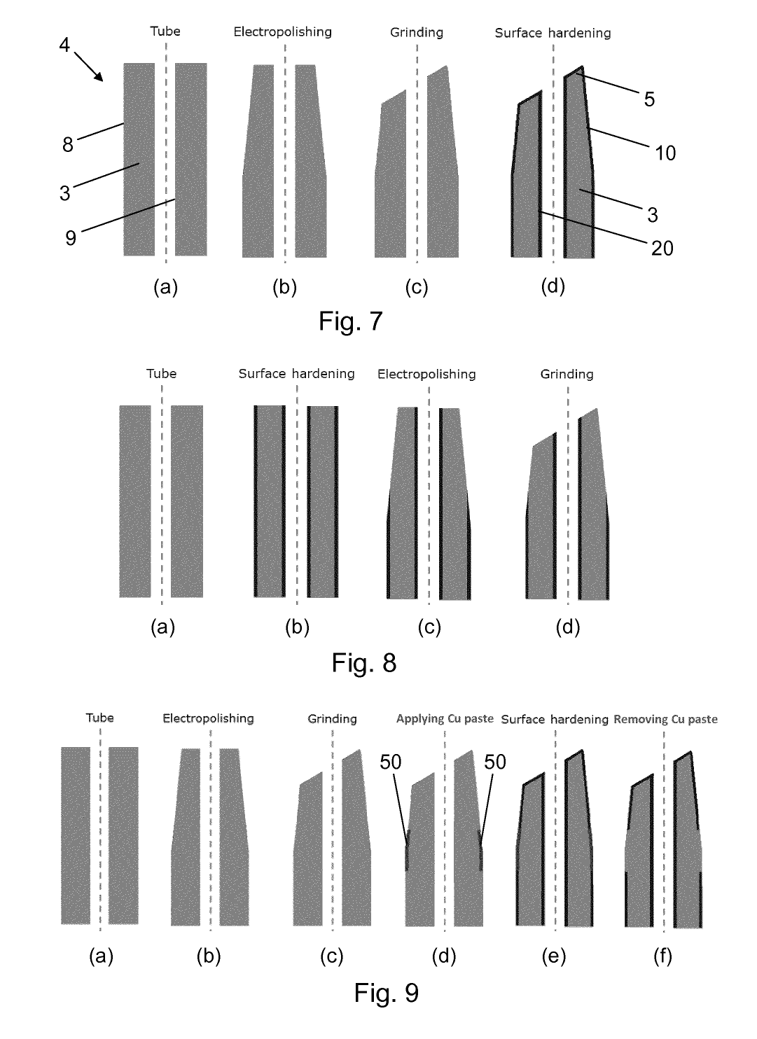

In accordance with the present invention, the injection needle 1 may undergo various pre- and/or post-surface hardening process steps to provide desired final properties and configuration. FIG. 7(a)-(d) shows an example of a pre-surface hardening processing of the injection needle 1, according to which the raw tubing is firstly electropolished to obtain a desired conical configuration of the subject end portion 4 and subsequently ground to obtain the sharpened tip 5. The final surface hardening of both the exterior surface 8 and the interior surface 9 yields an outer hardened surface layer 10, respectively an inner hardened surface layer 20, which provides a very hooking resistant injection needle.

FIG. 8(a)-(d) shows an example of a post-surface hardening processing of the injection needle 1, according to which the raw tubing is firstly electropolished to obtain a desired conical configuration of the subject end portion 4 and subsequently ground to obtain the sharpened tip 5. During the electropolishing and the grinding portions of the hardened surface layer are removed from the subject end portion 4, resulting in a hooking resistant injection needle which is less resistant to material wear than the injection needle of FIG. 7(d), but which is instead more pliable, thus being more capable of bending without breaking.

FIG. 9(a)-(f) shows an example of a combined pre- and post-surface hardening processing of the injection needle 1. In this example the raw tubing is firstly electropolished and ground. Thereafter, a Cu paste is applied to predefined portions of the exterior surface 8 before the injection needle 1 undergoes the surface hardening process. After the surface hardening the Cu paste is removed from the portions of the exterior surface 8, providing an injection needle 1 where areas of the tubular wall 3 are surface hardened while other areas are not. Thereby, a hooking resistant injection needle 1 is provided which exhibits greater flexibility in selected areas than in other areas. This is particularly usable when the injection needle 1 is intended to form part of an injection needle assembly, such as a pen needle assembly, as described in the below.

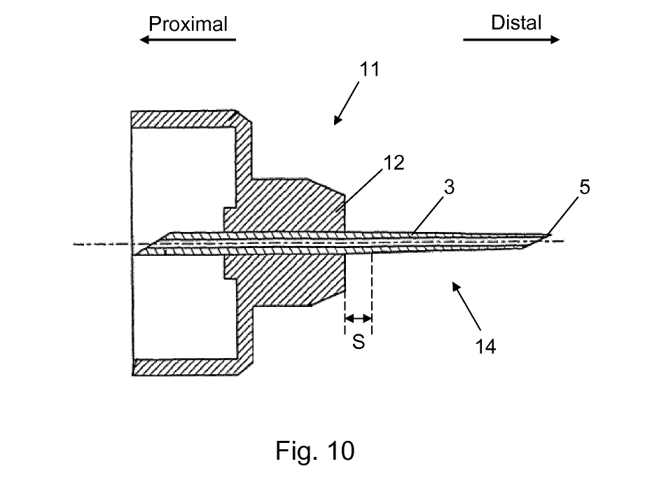

FIG. 10 is a longitudinal section view of the injection needle 1 as part of a pen needle assembly 11. The tubular wall 3 is fixed in a needle hub 12 such that a front needle 14 comprising the sharpened tip 5 extends distally therefrom for penetration of a skin membrane. A distal portion of the front needle 14 is surface hardened according to the present invention. Notably, the portion of the tubular wall 3 in the immediate vicinity of the needle hub 12 was covered by a mask during the surface hardening process to retain flexibility in that particular area. Specifically, the mask was applied around the tubular wall 3 in the area denoted by S in FIG. 10. This area is the most critical portion of the front needle 14 in terms of likelihood of breakage during insertion through skin. The fact that no surface hardening took place there reduces the risk of the tubular wall 3 breaking instead of just bending in response to a significant lateral force being applied to the sharpened tip 5.

The particular arrangement of the masking depends on the pen needle assembly model and the length of the front needle 14. If, for example, a 4 mm front needle is employed the mask may be arranged to cover an area of the tubular wall 3 which is between 1.5 mm and 5 mm, or between 2 mm and 5 mm, from the sharpened tip 5. If, alternatively, an 8 mm front needle is employed the mask may be arranged to cover an area of the tubular wall 3 which is between 1.5 mm and 9 mm, or between 3 mm and 9 mm, from the sharpened tip 5.

1. A medical injection needle having a metallic needle body comprising:
an axially extending wall,
a first end portion,
a second end portion, and
a flow path providing for fluid communication between the first end portion and the second end portion along the axially extending wall,
wherein at least a portion of the metallic needle body comprises a hardened surface layer in which carbon atoms and nitrogen atoms are deposited.

2. A medical injection needle according to claim 1, wherein the hardened surface layer has a radial extent (rₒ, rᵢ) which does not exceed 25 μm.

3. A medical injection needle according to claim 1, wherein the hardened surface layer has a radial extent (rₒ, rᵢ) which does not exceed 10 μm.

4. A medical injection needle according to claim 1, wherein the hardened surface layer comprises an inner layer in which predominantly carbon atoms are deposited and an outer layer in which predominantly nitrogen atoms are deposited.

5. A medical injection needle according to claim 1, wherein the at least a portion of the metallic needle body comprises the first end portion.

6. A medical injection needle according to claim 1, wherein the first end portion comprises a sharpened tip.

7. A medical injection needle according to claim 1, wherein the metallic needle body comprises a radially outwardly oriented surface and a radially inwardly oriented surface, and
wherein the hardened surface layer is present along at least a portion of at least one of the radially outwardly oriented surface and the radially inwardly oriented surface.

8. A medical injection needle according to claim 7, wherein the axially extending wall is tubular and has a thickness (t) in the range 25 μm to 50 μm.

9. A medical injection needle according to claim 7, wherein a first hardened surface layer is present along at least a portion of the radially outwardly oriented surface and a second hardened surface layer is present along at least a portion of the radially inwardly oriented surface,
wherein the metallic needle body further comprises a core section between the first hardened surface layer and the second hardened surface layer, and
wherein the hardness of the at least a portion of the radially outwardly oriented surface is 3-5 times greater than the hardness of the core section.

10. A medical injection needle according to claim 1, forming part of an injection needle assembly further comprising a needle hub element being adapted for coupling to an injection device,
wherein the metallic needle body is fixedly arranged in the needle hub element such that a portion of the axially extending wall extends distally from the needle hub element to define a front needle portion for insertion into human skin, and
wherein the hardened surface layer is only present along a surface which is further away from the needle hub element than 1.5 mm.

11. A method of hardening a medical injection needle having a metallic needle body comprising a longitudinal wall extending axially between a first end portion and a second end portion, the method comprising:
(i) bringing at least a portion of the medical injection needle having a first temperature, T₁, in the range 200° C. to 500° C. in contact with a gaseous substance derived from a compound containing nitrogen and carbon and having a second temperature, T₂, in the range 200° C. to 500° C.,
(ii) subsequent to (i) bringing the at least a portion of the medical injection needle in contact with a carbon gas having a third temperature, T₃, in the range T₁ to 500° C., and
(iii) subsequent to (ii) bringing the at least a portion of the medical injection needle in contact with a nitrogen gas having a fourth temperature, T₄, in the range T₁ to 500° C.

12. A method according to claim 11, further comprising:
(iv) prior to (i) electropolishing and/or grinding the first end portion.

13. A method according to claim 11, further comprising:
(v) subsequent to (iii) electropolishing and/or grinding the first end portion.

14. A method according to any of claim 11, further comprising:
(vi) prior to (i) covering a portion of the metallic needle body with a shield.

15. A method according to claim 14, wherein the first end portion comprises a sharpened tip, and wherein step (vi) comprises applying the shield at least around the metallic needle body in an area which is between 1.5 mm and 9 mm from the sharpened tip.
